(19) United States
(12) Patent Application Publication
(10) Pub. No.: US 2017/0079756 A1
(43) Pub. Date: Mar. 23, 2017

(54) HANDS-FREE DENTAL SUCTION ASSEMBLY

(71) Applicant: Karrie Lynn Velky, Ely, IA (US)

(72) Inventor: Karrie Lynn Velky, Ely, IA (US)

(21) Appl. No.: 15/267,998

(22) Filed: Sep. 16, 2016

Related U.S. Application Data
(60) Provisional application No. 62/219,827, filed on Sep. 17, 2015.

(51) Int. Cl. A61C 17/06; A61M 1/00

(52) U.S. Cl. CPC A61C 17/043; A61M 1/0086; A61M 2209/088

(57) ABSTRACT

A hands-free dental suction assembly having a pad made of an foam material. A pair of slits in spaced parallel relation are cut through an interior portion of the pad. A suction tip is threaded through the slits to connect the suction tip to the pad.

### CROSS-REFERENCE TO RELATED APPLICATIONS

This application claims priority to Provisional Application U.S. Ser. No. 62/219,827 filed on Sep. 17, 2015, which is herein incorporated by reference in its entirety.

### BACKGROUND OF THE INVENTION

This invention is directed to dental suction assembly, and more particularly to a hands-free dental suction assembly.

Use of a suction device for performing dental procedures is well-known in the art. The use of a suction device is frequently used with an ultrasonic scaler or any procedure where a dry-field is needed continually. Often, suction is usually provided by a dental assistant. As a result, the process is labor intensive and costly. Also, unless a barrier is placed between the suction tip and the patient's cheek, cheek tissue may be sucked into the tip stopping suction and damaging tissue. Finally, most suction tips obstruct a technician's view during a procedure. Therefore, a need exists in the art for a device that addresses these deficiencies.

Therefore, an objective of the present invention is to provide a dental suction assembly that can be used without hands.

A further objective of the present invention is to provide a dental suction assembly that improves suction and reduces tissue damage.

A still further objective is to provide a dental suction assembly that reduces the obstruction of a technician's view during a procedure.

These and other objectives will be apparent to those skilled in the art based upon the following written description, drawings, and claims.

### BRIEF DESCRIPTION OF THE FIGURES

FIG. 1 is a top plan view of a pad for a suction assembly;

FIG. 2 is a top plan view of a pad for a suction assembly;

FIG. 3 is a top plan view of a suction assembly; and

FIG. 4 is an end sectional view of a pad with adhesive material and a cover tab.

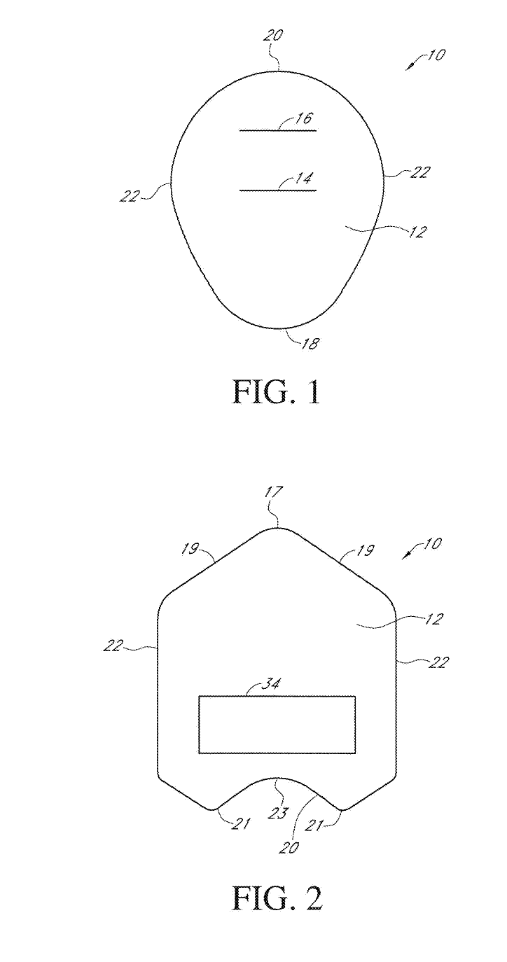

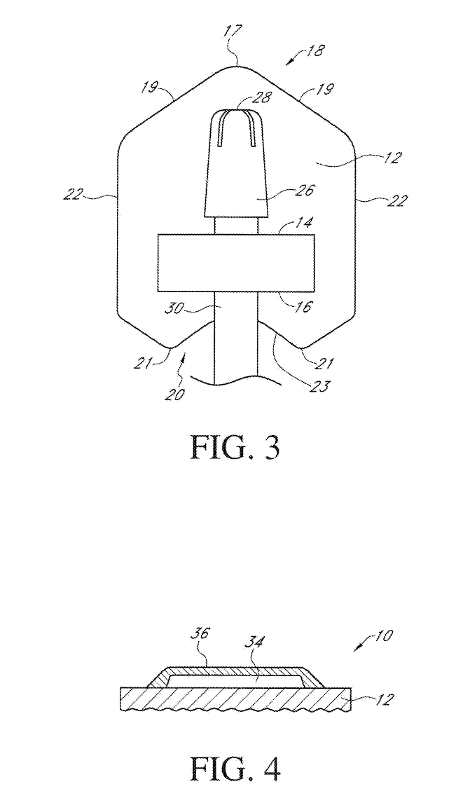

### DETAILED DESCRIPTION OF THE PREFERRED EMBODIMENTS

Referring to the figures. a hands-free dental suction assembly 10 includes a pad 12 having a pair of parallel slits 14 and 16. The pad 12 is of any type and preferably is made of a thin foam material having a generally triangular or egg shape and formed to comfortably cover the buccal mucosa of the cheek. As an example, the pad is made of a white foam such as one manufactured by Laminin Medical Products or an F-217®-3 manufactured by Foshan Osaka Export Co., Ltd. Alternatively, the pad is made of a three-ply co-extruded liner having a foam low density polyethylene (LDPE) core sandwiched between two layers of solid LDPE. The pad 10 has a first end 18 a second end 20, and side edges 22.

Preferably, slits 14 and 16 are an inch apart and centered in the middle of the pad along line 24 that extends from the first end 18 to the second end 20. Slit 16 is preferably ¼ inch from second end 20 and slits 14 and 16 are preferably ⅜ inch apart. The slits 14 and 16 preferably are ½ inch long. Alternatively, slit 16 is ⅜ inches from slit 16. Further, the pad 12 is preferably 1 ⅜ inches wide between side edges 22 and is 1 ⅝ inches long from end 18 to end 22. The size and shape of the pad 12 is important in order to keep suction tip in corner of the mouth while still providing comfort and minimizing obstruction.

In an alternative embodiment, the pad 12 has an apex 17 at end 18 and angled or tapered edges 19 that extend from the apex 17 to side edges 22. The bottom end 20 has a pair of outwardly extending convex edges 21 with a concave edge 23 that extends between convex edges 21.

Removably attached to the pad 12 is disposable and flexible SVE suction tip 26. The suction tip is threaded through slits 14 and 16 so that the pad 12 is connected to the suction tip 26.

In operation, the suction tip 26 is bent until the suctioning end 28 is parallel to the suctioning tube 30 without kinking the tube 30. The suction tip 26 is then threaded through slits 14 and 16 all the way to the end, of the suction tip 26. The suction tip 26 is then connected to an evacuation system.

With the patient's mouth open, the patient's cheek is pulled taught and assembly 10 is placed in the patient's mouth. The pad 12 is placed closest to the patient's cheek and is tucked between the patient's teeth and cheek. The tip or end 28 of the suction tip is placed as far back in the patient's mouth without causing discomfort. The patient turns their head toward the assembly 10 to help water pool at the suction tip 26 and be evacuated. The assembly 10 drapes at the corner of the patient's mouth and by closing their lips around the suction tube 30, more water than needed may be evacuated. When finished, the pad 12 is torn from the suction tip 26 and discarded.

In an alternative embodiment, the pad 12 is made of plastic. On one side is a latex free adhesive material 34 with a removable tab 36. When placed in a patient's mouth, the side with the adhesive material 34 faces away from the patients cheek. The suction tip 26 is attached to the adhesive 34 after the tab 36 is removed by wrapping the adhesive material around the suction tip 26. The assembly preferably is used by a dental professional for dental procedures such as regular prophylaxis, ultrasonic scaling, scaling and root planning, sealants, fillings, crown and bridge, veneers, and the like.

### SUMMARY OF THE INVENTION

A hands-free dental suction assembly having a pair of parallel slits positioned between a first and a second end. A flexible suction top is connected to the pad by threading the tip through the slits.

Alternatively, the assembly has an adhesive material on one side. The adhesive material is covered with a tab. The suction tip is attached to the adhesive material once the cover tab is removed.

What is claimed:

1. A hands-free dental suction assembly, comprising:
a pad having a pair of parallel slits positioned between a first end and a second end; and
a flexible suction tip threaded through the parallel slits to connect the pad to the suction tip.

2. The assembly of claim 1 wherein the parallel slits are between 3/8 and one inch apart and centered in a middle of the pad.

3. The assembly of claim 1 wherein one of the parallel slits is between and 3/8 inches from the second end of the pad.

4. The assembly of claim 1 wherein the parallel slits are ½ inch long.

5. The assembly of claim 1 wherein the pad is 1 ⅜ inches wide and 1 ⅝ inches long.

6. The assembly of claim 1 wherein the first end of the pad has an apex with a pair of angled edges and the second send has a pair of convex edges and a concave edge between the convex edges.

7. The assembly of claim 1 wherein the pad is made of a thin foam material.

8. A hands-free dental suction assembly, comprising:
a pad having a first side and a second side:
the first side having a latex free adhesive material with a removable tab: and
a flexible suction tip attached to the adhesive material after the tab is removed.
